(19) United States
(12) Patent Application Publication
(10) Pub. No.: US 2013/0131376 A1
(43) Pub. Date: May 23, 2013

(54) METHOD FOR PRODUCING A CARBOXYLIC ACID ESTER

(71) Applicants: Carlos Eduardo Marenco, Campinas (BR); Sergio Martins, Campinas (BR); Renato Bresciani, Campinas (BR); Joël Schwartz, Caluire (FR)

(72) Inventors: Carlos Eduardo Marenco, Campinas (BR); Sergio Martins, Campinas (BR); Renato Bresciani, Campinas (BR); Joël Schwartz, Caluire (FR)

(21) Appl. No.: 13/636,607

(22) PCT Filed: Mar. 21, 2011
(86) PCT No.: PCT/IB2011/000596

§ 371 Date: Nov. 21, 2012

(30) Foreign Application Priority Data
Mar. 25, 2010 (FR) 10 52163

(51) Int. Cl. C07C 67/58

(52) U.S. Cl. CPC C07C 67/58
USPC 560/248; 422/256

(57) ABSTRACT

A method for producing a carboxylic acid ester is described, particularly an acetic acid ester and specifically an ethyl acetate ester. Also described, is a method by which a carboxylic acid ester goes from an organic phase produced from an esterification reaction between a carboxylic acid and an alcohol. The organic phase can include, at least predominantly, the carboxylic acid ester and also minor amounts of alcohol and water. Further described, is a separation step, during which the organic phase is depleted of alcohol and free of carboxylic acid via a liquid-liquid extraction operation that is performed by placing said organic phase in contact with an aqueous phase, thus enabling the alcohol and the carboxylic acid, that are present in the organic phase, to be extracted to the aqueous phase.

The present invention relates to a process for producing a carboxylic acid ester.

The invention is targeted in particular at the production of acetic acid esters and more particularly ethyl acetate.

Acetic acid esters, in particular ethyl acetate, are generally used as organic solvents. In particular, ethyl acetate is used especially in the cosmetics and fragrance fields, and in adhesives, paints and varnishes.

Depending on the application targeted, a higher or lower purity is required and it is common to request that the amount of acetic acid present in the ethyl acetate be less than 0.01% by weight.

Thus, the processes for producing ethyl acetate must result in a quality product. Given that ethyl acetate is a standard, high-volume, consumer product, it is important that its production process be as effective as possible, in terms of productivity and energy balance.

Conventional processes for the manufacture of ethyl acetate provide an esterification stage coupled to a distillation stage, as described, for example, in patent GB 1 173 089. It may also be a reactive distillation, as described in application EP 1 220 829.

The reaction/distillation stages are generally followed by a stage of separation of the organic and aqueous phases resulting from the distillation top, as described in U.S. Pat. No. 4,314,947.

In order to meet the quality requirements of the ethyl acetate, U.S. Pat. No. 1,400,849 describes the subsequent purification of the ethyl acetate in order to separate the residual ethanol by distillation.

However, such processes consume energy and there exists a need to reduce production energy costs in this field.

The objective of the present invention is thus to provide a process for producing a carboxylic acid ester which is improved in terms of process economics.

With this aim, a subject matter of the present invention is a process for producing a carboxylic acid ester from an organic phase resulting from an esterification reaction between a carboxylic acid and an alcohol, said organic phase comprising at least predominantly the carboxylic acid ester and to a minor extent the alcohol, water and traces of carboxylic acid, characterized in that it comprises a separation stage during which the organic phase is depleted in alcohol and freed from the carboxylic acid by a liquid/liquid extraction operation by bringing said organic phase into contact with an aqueous phase which makes it possible to extract the alcohol and the carboxylic acid present in the organic phase toward the aqueous phase.

In the present text, the term “a phase comprising predominantly a given compound” is understood to mean a phase in which the given compound represents at least 85% of its weight, preferably at least 90% of its weight.

The term “a phase comprising to a minor extent a given compound” is understood to mean a phase in which the given compound represents less than 15% of its weight, preferably less than 10% of its weight.

The term “traces” is understood to mean less than 0.02% by weight, in particular between 0.01 and 0.02% by weight, of the compound concerned.

The term “depleted in alcohol” is understood to mean that at least 10% by weight of the alcohol has been extracted from the organic phase toward the aqueous phase, preferably at least 20% by weight and more preferably still at least 30% by weight.

The term “freed from the carboxylic acid” or “devoid” is understood to mean that the organic phase comprises less than 0.01% by weight, in particular between 0.001 and 0.01% by weight, of carboxylic acid.

Within the meaning of the present invention, the percentages by weight are expressed as percentage by weight with respect to the total weight of the phase (or stream) concerned.

In accordance with the process according to the invention, the organic phase is depleted in alcohol and freed from the carboxylic acid by a liquid/liquid extraction operation.

The liquid/liquid extraction consists in selectively separating one or more compounds of a mixture on the basis of chemical or physical properties. It concerns the extraction of a substance which is dissolved in a solvent, using another solvent, known as extraction solvent. The physical principle is the difference in solubility of the product to be extracted between the two liquid phases.

In the case of the present invention, the alcohol present in the organic phase, comprising predominantly the carboxylic acid ester and to a minor extent the alcohol, water and traces of carboxylic acid, is extracted with an aqueous phase not comprising the compounds to be extracted and in particular the alcohol. Preferably, the aqueous phase is predominantly composed of water; advantageously, it is pure water.

The aqueous phase and the organic phase are preferably introduced countercurrentwise, for example into a liquid/liquid extractor operating continuously.

On the one hand, an organic phase enriched in carboxylic acid ester, depleted in alcohol and freed from the carboxylic acid is obtained and, on the other hand, an aqueous phase comprising predominantly water, alcohol and traces of carboxylic acid is obtained.

According to a preferred embodiment of the invention, the organic phase enriched in carboxylic acid ester, depleted in alcohol and freed from the carboxylic acid has the following composition:
from 85 to 98% by weight, preferably from 90 to 95% by weight, of carboxylic acid ester,
from 0.5 to 3% by weight, preferably from 1 to 2% by weight, of alcohol,
from 2 to 8% by weight, preferably from 3 to 7% by weight, of water.

According to a preferred embodiment of the invention, the aqueous phase comprising predominantly water, alcohol and traces of carboxylic acid has the following composition:
from 85 to 98% by weight, preferably from 90 to 95% by weight, of water,
from 2 to 6% by weight, preferably from 3 to 5% by weight, of alcohol,
from 2 to 8% by weight, preferably from 4 to 6% by weight, of carboxylic acid ester,
traces of carboxylic acid.

The liquid/liquid extraction operation can be carried out at a temperature of between 15 and 50° C., preferably between 20 and 40° C.

Advantageously, the liquid/liquid extraction operation is carried out at atmospheric pressure or at a slightly lower or greater pressure, for example at a pressure of between 0.5 and 5 bar absolute.

According to the invention, the ratio of the flow rate of the organic phase to the flow rate of the aqueous phase at the start of the liquid/liquid extraction stage is between 2 and 10, preferably between 4 and 8. This ratio is optimized in order to reduce the overall energy consumption.

The number of theoretical stages of the liquid/liquid extractor is also optimised, so as to reduce by at least 10%, preferably by at least 20% and in particular by at least 30%, the amount of alcohol present in the organic phase which is subjected to the liquid/liquid extraction operation.

According to the process of the invention, the liquid/liquid extraction stage generally makes it possible to purify the carboxylic acid ester of at least 1%, preferably of 1 to 2% (absolute).

According to a first embodiment of the invention, the organic phase comprising predominantly the carboxylic acid ester and to a minor extent the alcohol, water and traces of carboxylic acid originates from an esterification process in the presence of a homogeneous or heterogeneous catalyst, followed by a distillation.

More specifically, the process comprises the following stages:
a) esterification reaction between the carboxylic acid and the alcohol, preferably in the presence of a homogeneous or heterogeneous catalyst,
b) distillation of the reaction medium resulting from stage a), making it possible to obtain, at the distillation column top, a vapor stream comprising predominantly the carboxylic acid ester and to a minor extent alcohol, water and traces of carboxylic acid,
c) condensation of the vapor stream resulting from stage b),
d) separation of the organic and aqueous phases, in particular by settling.

In accordance with an embodiment of the process of the invention, an esterification reaction of the carboxylic acid a) is carried out by an alcohol, preferably in the presence of an acid catalyst.

The carboxylic acid to be esterified is advantageously chosen from aliphatic carboxylic acids having from 1 to 6 carbon atoms; preferably, it is acetic acid.

The carboxylic acid to be esterified is advantageously introduced pure or in highly concentrated aqueous solution. The process of the invention does not exclude the presence of water in the carboxylic acid. However, it is preferable to use pure carboxylic acid due to the subsequent need to remove the water present in the carboxylic acid ester obtained at the end of the process.

The alcohol involved is preferably an alcohol comprising a linear or branched alkyl chain having from 1 to 6 carbon atoms or an alcohol comprising a cycloalkyl chain having 5 or 6 carbon atoms.

Preference is given in particular to alcohols comprising a low boiling point, in particular of less than 170° C., preferably of less than 165° C.

Thus, the alcohol comprising an alkyl chain is advantageously chosen from ethanol, butanol or n-propanol, preferably ethanol.

The alcohol comprising a cycloalkyl chain is preferably cyclohexanol.

According to the invention, the carboxylic acid can be introduced in deficit, in excess or in a stoichiometric amount, with respect to the alcohol.

An excess of one of the reactants can be advantageous in order to shift the equilibrium of the reaction toward the production of carboxylic acid ester.

Thus, the molar ratio of the carboxylic acid to the alcohol can be between 0.9 and 25, preferably between 1 and 20.

Preference is generally given to an excess of carboxylic acid with respect to the alcohol, preferably with a molar ratio of the carboxylic acid to the alcohol of between 7 and 25, in particular between 9 and 20.

The upper limit, for economic reasons, is advantageously chosen to be less than 25. The ratio precisely defined corresponds to the molar ratio of the reactants at the start of the reaction.

The catalyst that takes part in the process of the invention is a protonic acid catalyst. It can be a homogeneous or heterogeneous catalyst.

According to a first form, the catalyst is a heterogeneous acid catalyst.

The heterogeneous acid catalysts of the invention are preferably sulfonic resins or zeolites.

The zeolites which can be used are, for example, those cited in the document WO 2007/099071.

The resins which are suitable for the present invention can be composed of a polystyrene or polyacrylic backbone which carries sulfonic functional groups.

Thus, use may be made of commercial sulfonic acid SO₃H or carboxylic acid COOH resins, which resins are sold under various trade names.

Mention may be made, inter alia, of the following esterification resins: Amberlyst® 15 from Rohm & Haas, Amberlite® IR-120H from Rohm & Haas, and Lewatit® 2631 and K1431 from Bayer.

The acidity of these resins is, for example, between 1 and 10 eq/kg (H+).

These resins are used in particular in a fixed or fluidized bed, preferably in a fixed bed.

According to a second form, the catalyst is a homogeneous acid catalyst.

The term “strong acid” denotes, in the present invention, an acid exhibiting a pKa in water of less than 2, preferably of less than 1.

The pKa is defined as follows: pKa=−log Ka, Ka being the ionic dissociation constant of the acid/base pair at ambient temperature (generally 25° C.), when water is used as solvent.

Among the acids corresponding to this definition, it is preferable to use an acid which does not result in side reactions harmful to the esterification process and which in particular does not exhibit an oxidizing nature, such as nitric acid.

Mention may more particularly be made, as homogeneous strong acid, of sulfuric acid, sulfonic acids and mixtures thereof.

Mention may in particular be made, as sulfonic acids, of fluorosulfonic acid, chlorosulfonic acid or trifluoromethanesulfonic acid, methanesulfonic acid, ethanesulfonic acid, camphenesulfonic acid, benzenesulfonic acid, toluenesulfonic acids, xylenesulfonic acids or naphthalenesulfonic acids.

Among these acids, the preferred catalyst is chosen from para-toluenesulfonic acid or methanesulfonic acid, preferably methanesulfonic acid.

The amount of catalyst introduced is preferably between 0.1 and 2% by weight, with respect to the reaction medium at the start of the reaction.

It is possible to introduce other compounds into the reaction, for example corrosion inhibitors. These may in particular be copper(II) sulfate.

In accordance with the process of the invention, the esterification reaction can be carried out according to a continuous or batchwise form.

According to a preferred embodiment of the invention, the process is a continuous process.

In the process of the invention, the carboxylic acid and the alcohol can be introduced alone or as a mixture, preferably as a mixture.

According to one embodiment of the invention, the esterification stage a) is carried out takes place in the presence of an acid catalyst at a temperature at least equal to 50° C.

Advantageously, the reaction temperature is between 50 and 150° C., preferably between 100 and 130° C.

The reaction is preferably carried out at atmospheric pressure. A pressure slightly greater than or lower than atmospheric pressure may also be suitable. Thus, the process of the invention can be carried out, for example, at an absolute pressure of between 0.5 and 5 bar absolute.

According to one embodiment of the invention, the reaction mixture obtained above in stage a) is subsequently subjected to a distillation operation b) in order to obtain, at the distillation top, a vapor stream comprising predominantly the carboxylic acid ester and to a minor extent the alcohol, water and traces of carboxylic acid.

The distillation operation b) is preferably carried out in a distillation column. The feed point where the reaction mixture is introduced is, in general, substantially at the mid-height of the distillation column. It can also be located lower down, at a height between the mid-height of the column and the bottom of the column.

The temperature at the distillation bottom is preferably between 50 and 150° C., preferably between 100 and 130° C.

The defined pressure at the distillation top is preferably between 0.5 and 5 bar absolute; advantageously, the distillation top pressure is between 1 and 2 bar absolute.

Advantageously, the distillation operation b) makes it possible to obtain:
a vapor stream at the distillation top comprising predominantly the carboxylic acid ester and to a minor extent alcohol and optionally water, and
a medium at the distillation bottom comprising predominantly carboxylic acid and to a minor extent alcohol and ester and optionally catalyst.

In the process of the invention, the vapor stream at the distillation top comprises in particular:
from 75 to 98% by weight of carboxylic acid ester,
from 0 to 17% by weight of water,
from 0 to 8% by weight of alcohol,
traces of carboxylic acid.

The vapor stream at the distillation top more preferably still comprises:
from 85 to 95% by weight of carboxylic acid ester,
from 0 to 10% by weight of water,
from 0 to 5% by weight of alcohol,
traces of carboxylic acid.

The term “traces of carboxylic acid” is understood to mean less than 0.02% by weight, preferably between 0.001 and 0.02% by weight, of carboxylic acid.

In the process of the invention, the medium at the distillation bottom comprises in particular:
from 60 to 98% by weight of carboxylic acid,
from 0 to 15% by weight of carboxylic acid ester,
from 0 to 10% by weight of water,
from 0 to 15% by weight of alcohol.

The medium at the distillation bottom preferably comprises:
from 80 to 95% by weight of carboxylic acid,
from 2 to 15% by weight of carboxylic acid ester,
from 2 to 8% by weight of water,
from 0.1 to 2% by weight of alcohol.

The medium at the distillation bottom can additionally comprise up to 2% by weight of catalyst.

According to this specific embodiment of the invention in which the esterification reaction a) is carried out in the presence of an excess of carboxylic acid, the medium at the distillation bottom is advantageously withdrawn and recycled by reintroduction upstream of or during the reaction a), preferably upstream.

The term “upstream” is understood to mean that the medium at the distillation bottom is reintroduced into the mixture of carboxylic acid and alcohol before it has reacted.

In this case, the ratio of the flow rate of the recycled stream to the feed flow rate of the reaction mixture (carboxylic acid+alcohol) is advantageously between 4 and 20, preferably between 5 and 15.

In this embodiment, the carboxylic acid/alcohol molar ratio is either the ratio in the mixture comprising the carboxylic acid and the alcohol before the reaction when the reactants are introduced as a mixture, or the ratio at the start of the reaction when the reactants are introduced separately into the reaction. This ratio takes into account the supply of carboxylic acid and of alcohol originating from the recycling. This carboxylic acid/alcohol molar ratio is preferably between 7 and 25 and more preferably between 9 and 20.

The vapor stream resulting from stage b) is advantageously condensed in the liquid form by lowering its temperature to a temperature, for example, of between 15° C. and 40° C. by passing it through one or more condensers.

The liquid stream thus obtained can subsequently be sent to a means for separation of liquid phases, preferably a decanter which separates the organic phase, comprising predominantly the carboxylic acid ester and to a minor extent the alcohol, water and traces of carboxylic acid, and the aqueous phase, comprising predominantly water and to a minor extent alcohol and carboxylic acid.

The organic phase is advantageously partly reintroduced at the distillation top in order to ensure that the column is operating under reflux.

The reflux ratio is preferably between 1 and 8, advantageously between 2 and 6.

In accordance with the invention, the remainder of the organic phase resulting from stage d), composed predominantly of the carboxylic acid ester and to a minor extent of alcohol, water and traces of carboxylic acid, is subsequently subjected to a liquid/liquid extraction stage as described above.

According to a second embodiment of the invention, the organic phase comprising predominantly the carboxylic acid ester and to a minor extent the alcohol, water and traces of carboxylic acid originates from a reactive distillation process.

More specifically, the process comprises the following stages:
a′) reactive distillation between the carboxylic acid and the alcohol, preferably in the presence of a catalyst, making it possible to obtain, at the distillation column top, a vapor stream comprising predominantly the carboxylic acid ester and to a minor extent alcohol, water and traces of carboxylic acid,
b′) condensation of the vapor stream resulting from stage a′),
c′) separation of the organic and aqueous phases, in particular by settling.

According to this specific form, according to which the esterification reaction and the distillation are carried out in just one and the same stage a′), the nature of the reactants involved can be identical to that of the reactants defined above and the top vapor stream has a similar composition.

The reactive distillation column comprises in particular at least one reaction region and at least one nonreaction region.

The packing body optionally used in the process of the present invention is chosen as a function of the efficiency necessary. The packing body can be chosen from the packing bodies well known to a person skilled in the art, such as, for example, solids in the form of rings, polylobal extrudates or saddles. Mention may be made, as nonlimiting examples of packing bodies, of Raschig rings, Pall rings, Intos rings, Berl saddles, Novalox saddles and Intalox saddles. However, the packing body can also be chosen from structured packings, for example of Flexipac (registered trade name) type, sold by Koch, or Sulzer Chemtech or Sulzer (registered trade names) type, sold by Sulzer.

The reaction region of the reactive distillation column generally comprises at least one esterification catalytic bed. The nonreaction region corresponds to the region devoid of catalyst, generally comprising a stacked packing, a random packing or plates.

The catalyst can be enclosed in at least one permeable casing, which casing is composed, for example, of a cloth made of fabric made of synthetic material, for example made of polypropylene, or made of metal fabric.

The catalyst can also be positioned randomly, that is to say freely, inside each catalytic bed of the catalytic region.

In this case, in order to keep the catalyst in place and to prevent it from being carried away by the stream of liquid which passes through it, provision is generally made for any catalytic bed included in the catalytic region to rest on any contrivance which allows the liquid to pass but which is impermeable to the catalytic particles, such as, for example, a screen of Johnson type or nozzles of Johnson type evenly distributed over a surface impermeable to gas, liquid or solid streams.

Advantageously, said contrivance is slightly raised with respect to the inlet of the substantially radial circulation means, over the bottom of the catalytic region, so as to create a distribution region situated partially below the catalytic bed of the catalytic region.

When the catalyst is positioned randomly, provision may be made to use it in the form of a fixed bed, expanded bed or fluidized bed. Whether the catalyst is positioned randomly or enclosed in at least one casing, the void fraction which can be conferred on it is generally between 30 and 70%.

As regards stage a′) of the process using a reactive distillation column, the following stages are generally carried out:
1) the alcohol and the carboxylic acid are fed to a distillation column comprising at least one reaction region and at least one nonreaction region;
2) the alcohol and the acid react in the reaction region or regions in the presence of a heterogeneous catalyst, and the compounds formed are separated by distillation.

The alcohol is preferably fed to the lower part of each reaction region or below each of these regions. The acid is preferably fed to the upper part of each reaction region or above each of these regions.

It is very particularly preferable for the reaction in stage 2) to be carried out so that the carboxylic acid/alcohol molar ratio is between 1 and 2, preferably between 1 and 1.5.

The proportion of heterogeneous catalyst in the reactive sections is preferably between 10 and 50% by volume.

The temperature at the reactive distillation bottom is preferably between 50 and 150° C., preferably between 100 and 130° C.

The defined pressure at the reactive distillation top is preferably between 0.5 and 5 bar absolute; advantageously, the pressure at the distillation top is between 1 and 2 bar absolute.

The vapor stream resulting from stage a′) is advantageously condensed in the liquid form by lowering its temperature to a temperature, for example, of between 15° C. and 40° C., by passing it through one or more condensers.

The liquid stream thus obtained can subsequently be sent to a means for separation of liquid phases, preferably a decanter which separates the organic phase, comprising predominantly the carboxylic acid ester and to a minor extent the alcohol, water and traces of carboxylic acid, and the aqueous phase, comprising predominantly water and to a minor extent alcohol and carboxylic acid.

The organic phase is, advantageously, partly reintroduced at the distillation top in order to ensure that the column is operating under reflux.

The reflux ratio is preferably between 0.5 and 5, advantageously between 0.5 and 2.

In accordance with the invention, the remainder of the organic phase resulting from stage c′), composed predominantly of the carboxylic acid ester and to a minor extent of alcohol, water and traces of carboxylic acid, is subsequently subjected to a liquid/liquid extraction stage as described above.

According to the process of the invention, the organic phase, comprising predominantly the carboxylic acid ester and to a minor extent the alcohol, water and traces of carboxylic acid, which results from stages d) or c′), is depleted in alcohol and freed from the carboxylic acid by a liquid/liquid extraction operation by bringing said organic phase into contact with an aqueous phase which makes it possible to extract the alcohol and the carboxylic acid present in the organic phase toward the aqueous phase.

According to a specific embodiment of the invention, the aqueous phase used for the liquid/liquid extraction can originate, in all or in part, from the aqueous phase resulting from the stage of separation of the phases d) or c′), this aqueous phase preferably having been subjected to a purification, for example by passing through an entrainment device, in particular a column for separation of the water and of the alcohol present in it.

At the outlet of the entrainment device, the phase comprising predominantly alcohol can advantageously be recycled by reintroduction at the start of the reaction and the phase comprising predominantly water can be used for the liquid/liquid extraction.

According to a preferred embodiment of the process of the invention, the organic phase depleted in alcohol and freed from the carboxylic acid recovered at the outlet of the liquid/liquid extraction stage is subjected to a distillation operation, for example in a distillation column. During this stage, the residual water and the residual alcohol, which could not be removed during the liquid/liquid extraction stage as described above in the present description, are removed and the purified carboxylic acid ester is recovered. The water and the alcohol are removed at the distillation top and the purified carboxylic acid ester is recovered at the distillation bottom.

The term “purified” within the meaning of the present invention is understood to mean that the carboxylic acid ester thus recovered comprises less than 0.1% by weight of alcohol, preferably between 0.015% and 0.100% by weight of alcohol, and less than 0.1% by weight of water, preferably between 0.010% and 0.100% by weight, and is devoid of carboxylic acid.

According to a preferred embodiment, the process of the invention comprises the following stages:
a) esterification reaction between the carboxylic acid and the alcohol, preferably in the presence of a catalyst,
b) distillation of the reaction medium resulting from stage a), making it possible to obtain, at the distillation column top, a vapor stream comprising predominantly the carboxylic acid ester and to a minor extent the alcohol, water and traces of carboxylic acid,
c) condensation of the vapor stream resulting from stage b),
d) separation of the organic and aqueous phases, in particular by settling,
e) liquid/liquid extraction of the organic phase resulting from stage d),
f) distillation of the organic phase resulting from stage e),
g) recovery of the purified carboxylic acid ester.

According to another preferred embodiment, the process of the invention comprises the following stages:
a′) reactive distillation between the carboxylic acid and the alcohol, preferably in the presence of a catalyst, making it possible to obtain, at the distillation column top, a vapor stream comprising predominantly the carboxylic acid ester and to a minor extent the alcohol, water and traces of carboxylic acid,
b′) condensation of the vapor stream resulting from stage a′),
c′) separation of the organic and aqueous phases, in particular by settling,
d′) liquid/liquid extraction of the organic phase resulting from stage c′),
e′) distillation of the organic phase resulting from stage d′),
f′) recovery of the purified carboxylic acid ester.

According to another more specific aspect, another subject matter of the present invention is a device for the implementation of the process of the invention.

This device, which is generally provided in the form of a plant of industrial dimensions, comprises a liquid/liquid extraction device, means for feeding the liquid/liquid extraction device with organic phase comprising predominantly the carboxylic acid ester and to a minor extent the alcohol, water and traces of carboxylic acid, and means for feeding the liquid/liquid extraction device with aqueous phase, and also means for recovery of the aqueous and organic phases at the outlet of the liquid/liquid extraction device.

Preferably, this device also comprises, upstream, a reactor, a distillation column, the top part of which is connected to means for treating the vapor stream resulting from the distillation column, namely at least one condenser, the outlet of which is connected to a liquid/liquid phase separator, preferably a decanter, itself connected to the liquid/liquid extraction device.

Alternatively, this device can also comprise, upstream, a reactive distillation column, the top part of which is connected to means for treating the vapor stream resulting from the reactive distillation column, namely at least one condenser, the outlet of which is connected to a liquid/liquid phase separator, preferably a decanter, itself connected to the liquid/liquid extraction device.

### DESCRIPTION OF THE FIGURES

The invention will also be explained in further detail by means of the following description, made with reference to FIGS. 1, 2 and 3.

FIG. 1 is a diagrammatic representation of a preferred device for the implementation of the process according to the invention comprising a reactor 1, a distillation column 2, the top part of which is connected to means for treating the vapor stream resulting from the distillation column 2, namely at least one condenser 4, the outlet of which is connected to a decanter 5.

The alcohol A and the carboxylic acid B form a stream (F₀) which is introduced at 0 into a reactor 1.

The reactor is preferably adiabatic. It can be of the perfectly stirred type or of the plug flow type, preferably of the plug flow type. The stream (F₁) resulting from the reaction is introduced at 1a into a distillation column 2.

This stage is targeted at obtaining, at the distillation top, a vapor stream (F₂) comprising predominantly the expected carboxylic acid ester and to a minor extent the alcohol, water and traces of carboxylic acid. At the distillation bottom, the liquid stream (F₃) comprises predominantly the carboxylic acid.

A person skilled in the art is fully in a position to choose the means to be employed depending on the separation to be carried out.

The following will simply be mentioned. The size (especially the diameter) of the distillation columns depends on the stream circulating and on the internal pressure. They will therefore be sized mainly according to the flow rate of mixture to be treated. The internal parameter which is the number of theoretical stages is determined in particular by the purity of the starting compound and the purities of the products which have to be obtained at the distillation top and bottom.

It will be specified that the column can be packed without distinction with plates or with stacked or woven packing, as is fully known to a person skilled in the art.

Once the plant has been determined, a person skilled in the art adjusts the operating parameters of the column.

Thus, the distillation column can advantageously, but not limitingly, be a column having the following specifications:
number of theoretical stages: from 1 to 40, preferably from 2 to 20,
reflux ratio R of between 1 and 8, preferably between 2 and 6.

The reflux ratio is defined as the ratio of the flow rate of material reinjected from the top of the column to the inside of the column to the flow rate of organic phase actually exiting from the decanter.

In order to carry out the distillation, heat can be supplied to the column bottom in particular by a shell-and-tube heat exchanger, a plate heat exchanger, a coil-type heat exchanger or any other equivalent device. Heating can be carried out using steam or a heat-exchange fluid.

A preferred embodiment consists in heating the mixture at the distillation bottom in a heat exchanger 3 by removing a stream (F₄) at the bottom which circulates in a loop. More specifically, the stream (F₃) exits at the distillation bottom and a fraction (F₄) passes through a heat exchanger from the bottom upward and, on exiting from the exchanger, is introduced in the form of a liquid/vapor mixture into the lower part of the distillation column.

Another embodiment consists in carrying out a forced circulation of the stream (F₃) in the exchanger using a pump.

The vapor stream (F₂) at the column top, comprising predominantly the carboxylic acid ester and to a minor extent the alcohol, water and traces of carboxylic acid, is condensed so as to recover a liquid stream, one fraction (F₆) of which is introduced as a sidestream at the column top, to ensure the reflux in the column, and the other fraction (F₉) of which is treated in a subsequent stage of purification of the carboxylic acid ester.

At the distillation top, the stream comprising predominantly the carboxylic acid ester and to a minor extent the alcohol, water and traces of carboxylic acid is recovered from the stream (F₂) by condensation, for example by passing through one or more condensers 4.

The vapor phase (F₂) is cooled and converted into liquid form via cooling by lowering its temperature to a temperature for example of between 15° C. and 40° C.

This operation is carried out by passing through a condenser, which is a conventional device, for example a tubular heat exchanger fed with a heat-exchange fluid (generally water) maintained at a temperature close to the chosen cooling temperature.

The number and size of the condensers are chosen as a function of the cooling capacities of the coolants circulating in the condensers.

In the case of condensers in series, the vapor phase exiting from the first condenser is introduced into the second condenser.

The liquid stream (F₇) is recovered at the outlet of the condenser(s).

The liquid stream (F₇) is introduced into a decanter 5 which separates the aqueous phase (F₆) from the organic phase (F₆+F₉).

The expected ester present in the stream (F₉) is sent to a liquid/liquid extraction device 6 fed, for example countercurrentwise, with a stream (F₁₀) of water C.

At the outlet of the liquid/liquid extractor 6, a stream (F₁₂), comprising predominantly water and to a minor extent the alcohol and traces of carboxylic acid, and a stream (F₁₁), comprising predominantly the carboxylic acid ester depleted in alcohol, in water and in carboxylic acid, are obtained, which stream (F₁₁) is subsequently sent to a distillation column 7 from which the following are recovered: at the distillation top, a stream (F₁₄) comprising residual amounts of alcohol and water and, at the distillation bottom, a stream (F₁₃) comprising the purified ester.

FIG. 2 is a diagrammatic representation of another preferred device for the implementation of the process according to the invention comprising a reactor 1, a distillation column 2, the top part of which is connected to means for treating the vapor stream resulting from the distillation column 2, namely at least one condenser 4, the outlet of which is connected to a decanter 5.

The alcohol A and the carboxylic acid B form a stream (F₀) which is introduced at 0 into a reactor 1.

The reactor is preferably adiabatic. It can be of the perfectly stirred type or of the plug flow type, preferably of the plug flow type. The stream (F₁) resulting from the reaction is introduced at 1a into a distillation column 2.

This stage is targeted at obtaining, at the distillation top, a vapor stream (F₂) comprising predominantly the expected carboxylic acid ester and to a minor extent the alcohol, water and traces of carboxylic acid. At the distillation bottom, the liquid stream (F₃) comprises predominantly the carboxylic acid.

The distillation column is advantageously, but not limitingly, a column having the following specifications:
number of theoretical stages: from 1 to 40, preferably from 2 to 20,
reflux ratio R of between 1 and 8, preferably between 2 and 6.

A preferred embodiment consists in heating the mixture at the distillation bottom in a heat exchanger 3 by removing a stream (F₄) at the bottom which circulates in a loop. More specifically, the stream (F₃) exits at the distillation bottom and a fraction (F₄) passes through a heat exchanger from the bottom upward and, on exiting from the exchanger, is introduced in the form of a liquid/vapor mixture into the lower part of the distillation column.

Another embodiment consists in carrying out a forced circulation of the stream (F₃) in the exchanger using a pump.

Advantageously, the stream (F₅) exiting from the exchanger is rerouted upstream of the reactor 1, for example via pumps. If the stream (F₅) is rerouted upstream of the reactor 1, then the ratio of the flow rate of the stream (F₅) to the flow rate of the stream (F₀) is preferably between 4 and 20.

The vapor stream (F₂) at the column top, comprising predominantly the carboxylic acid ester and to a minor extent the alcohol, water and traces of carboxylic acid, is condensed so as to recover a liquid stream, one fraction (F₆) of which is introduced as a sidestream at the column top, to ensure the reflux in the column, and the other fraction (F₉) of which is treated in a subsequent stage of purification of the carboxylic acid ester.

At the distillation top, the stream comprising predominantly the carboxylic acid ester and to a minor extent the alcohol, water and traces of carboxylic acid is recovered from the stream (F₂) by condensation, for example by passing through one or more condensers 4.

The vapor phase (F₂) is cooled and converted into liquid form via cooling by lowering its temperature to a temperature for example of between 15° C. and 40° C.

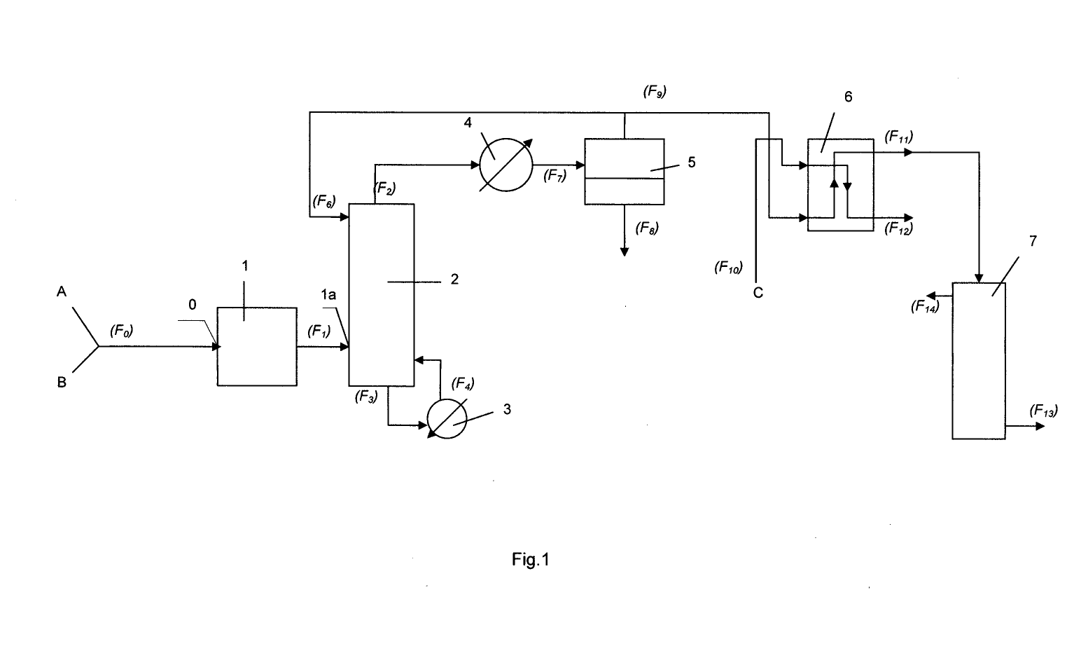

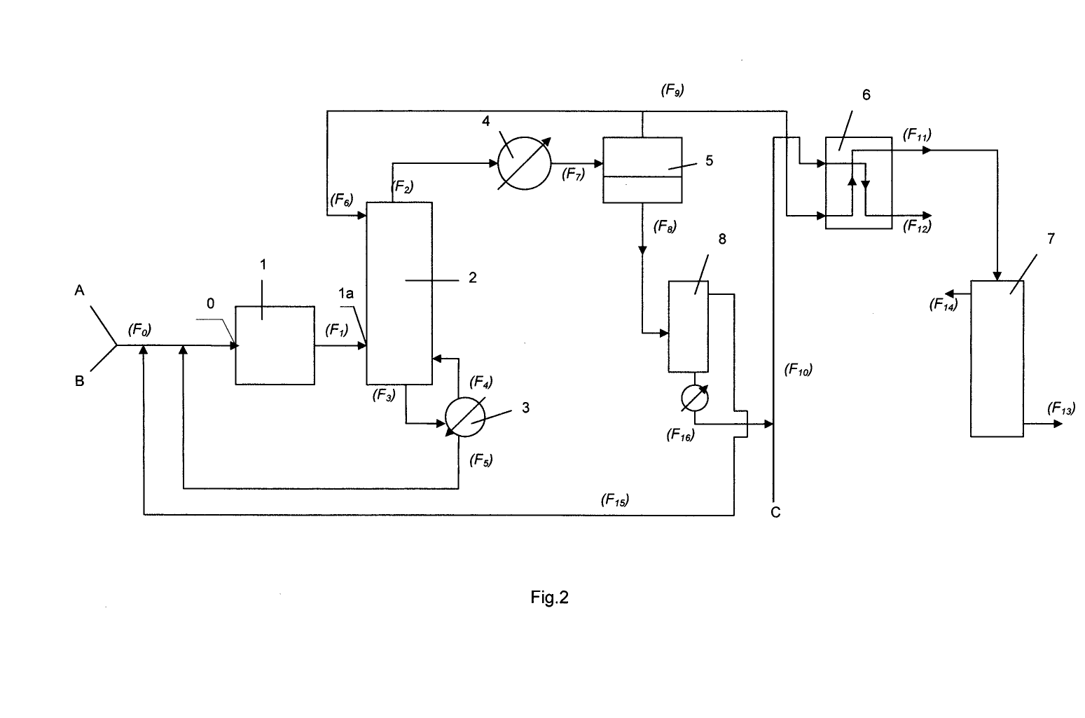

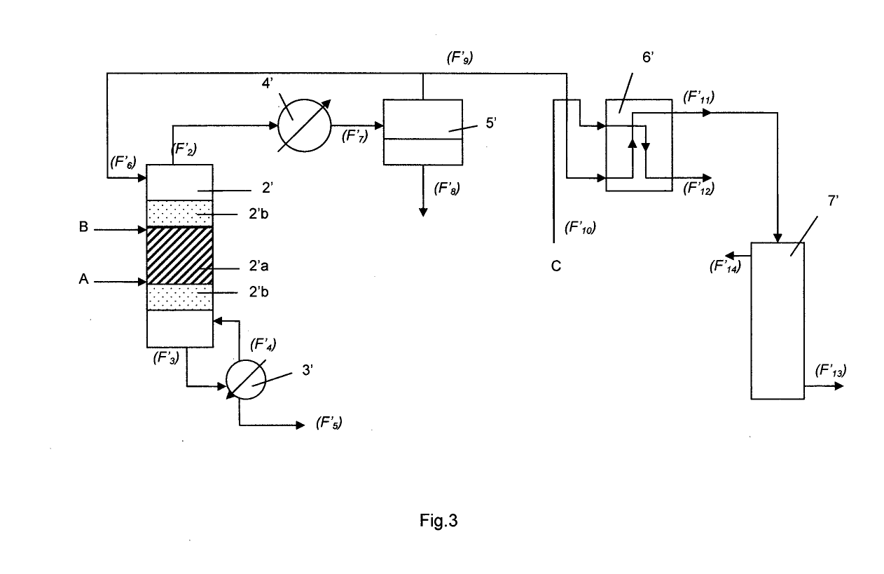

This operation is carried out as described for FIG. 1.

The liquid stream (F₇) is recovered at the outlet of the condenser(s).

The liquid stream (F₇) is introduced into a decanter 5 which separates the aqueous phase (F₆) from the organic phase (F₆+F₉).

The phase (F₆) can be retreated by passing through an entrainment device 8 which makes it possible to recover, on the one hand, a phase comprising predominantly water (F₁₆) and, on the other hand, a phase comprising predominantly alcohol and carboxylic acid ester as a mixture (F₁₅). The stream (F₁₅) can be rerouted upstream of the reactor 1, for example via pumps.

The expected ester present in the stream (F₉) is sent to a liquid/liquid extraction device 6 fed, for example countercurrentwise, with a stream (F₁₀) of water C, which can originate, in all or in part, from the stream (F₁₆).

At the outlet of the liquid/liquid extractor 6, a stream (F₁₂), comprising predominantly the water and to a minor extent the alcohol and traces of carboxylic acid, and a stream (F₁₁), comprising predominantly the carboxylic acid ester depleted in alcohol, in water and in carboxylic acid, are obtained, which stream (F₁₁) is subsequently sent to a distillation column 7 from which the following are recovered: at the distillation top, a stream (F₁₄) comprising residual amounts of alcohol and water and, at the distillation bottom, a stream (F₁₃) comprising the purified ester.

FIG. 3 is a diagrammatic representation of another preferred device for the implementation of the process according to the invention comprising a reactive distillation column 2′, the top part of which is connected to means for treating the vapor stream resulting from the reactive distillation column 2′, namely at least one condenser 4′, the outlet of which is connected to a decanter 5′. The reactive distillation column comprises a reaction region 2′ a comprising a catalytic bed and two nonreaction regions 2′ b on either side of the reaction region devoid of catalytic bed and consisting, for example, of a stacked packing, a random packing or plates.

The alcohol A and the carboxylic acid B are introduced separately into the reactive region 2′ a of the reactive distillation column 2′, the carboxylic acid being introduced above the alcohol.

There is obtained, at the top of the reactive distillation column 2′, a vapor stream (F′₂) comprising predominantly the carboxylic acid ester and to a minor extent the alcohol, water and traces of carboxylic acid. At the distillation bottom, the liquid stream (F′₃) comprises predominantly water, which water is drawn off at the reboiler (F′₅).

The reactive distillation column can advantageously, but not limitingly, be a column having the following specifications:
number of theoretical stages: from 1 to 40, preferably from 2 to 20,
reflux ratio R of between 0.5 and 5, preferably between 0.5 and 2.

In order to carry out the distillation, heat can be supplied to the column bottom in particular by a shell-and-tube heat exchanger, a plate heat exchanger, a coil-type heat exchanger or any other equivalent device. Heating can be carried out using steam or a heat-exchange fluid.

A preferred embodiment consists in heating the mixture at the distillation bottom in a heat exchanger 3′ by removing a stream (F′₄) at the bottom which circulates in a loop. More specifically, the stream (F′₃) exits at the distillation bottom and a fraction (F′₄) passes through a heat exchanger from the bottom upward and, on exiting from the exchanger, is introduced in the form of a liquid/vapor mixture into the lower part of the distillation column.

Another embodiment consists in carrying out a forced circulation of the stream (F′₃) in the exchanger using a pump.

The vapor stream (F′₂) at the column top, comprising predominantly the carboxylic acid ester and to a minor extent the alcohol, water and traces of carboxylic acid, is condensed so as to recover a liquid stream, one fraction (F′₆) of which is introduced as a sidestream at the column top, to ensure the reflux in the column 2′, and the other fraction (F′₉) of which is treated in a subsequent stage of purification of the carboxylic acid ester.

At the distillation top, the stream comprising essentially the carboxylic acid ester and to a minor extent the alcohol, water and traces of carboxylic acid is recovered from the stream (F′₂) by condensation, for example by passing through one or more condensers 4′.

The vapor phase (F′₂) is cooled and converted into liquid form via cooling by lowering its temperature to a temperature for example of between 15° C. and 40° C.

This operation is carried out as described for FIG. 1.

The liquid stream (F′₇) is recovered at the outlet of the condenser(s).

The liquid stream (F′₇) is introduced into a decanter 5′ which separates the aqueous phase (F′₆) from the organic phase (F′₆+F′₉).

The expected ester present in the stream (F′₉) is sent to a liquid/liquid extraction device 6′ fed, for example countercurrentwise, with a stream (F′₁₀) of water C.

At the outlet of the liquid/liquid extractor 6′, a stream (F′₁₂), comprising predominantly the water and to a minor extent alcohol, and a stream (F′₁₁), comprising predominantly the carboxylic acid ester depleted in alcohol, in water and in carboxylic acid, are obtained, which stream (F′₁₁) is subsequently sent to a distillation column 7′ from which the following are recovered: at the distillation top, a stream (F′₁₄) comprising residual amounts of alcohol and water and, at the distillation bottom, a stream (F′₁₃) comprising the purified ester.

### ADVANTAGES

The process of the invention is particularly interesting due to the advantages which it provides.

One of the advantages of the process of the present invention is that less energy is consumed.

This is because the presence of the liquid/liquid extraction stage renders the second distillation of the organic phase resulting from the liquid/liquid extraction stage much more economic from the energy viewpoint.

The following examples illustrate the invention without, however, limiting it.

### EXAMPLES

For better understanding, the examples below are described with reference to FIG. 2. The following examples are given for a final production of 100 kg/h of ethyl acetate.

### Example 1 According to the Invention (E-1)

An esterification reactor 1 is fed by simultaneous introduction of ethanol A with a flow rate of 56 kg/h and of acetic acid B with a flow rate of 69 kg/h. The total feed flow rate is referred to as flow rate (F₀).

2.3 kg of methanesulfonic acid are also introduced into the esterification reactor 1 as catalyst. The esterification reactor 1 has a capacity of 230 kg.

Methanesulfonic acid is regularly injected and bled off, so as to maintain the catalytic activity.

The inlet temperature of the reactor is 117° C. The reactor is adiabatic.

The outlet stream (F₁) of the esterification reactor subsequently feeds an esterification column 2. This column comprises 16 theoretical plates. It operates at a pressure of 1.5 bar absolute and at a column bottom temperature of 118° C.

The stream (F₅) originating from the bottom of the esterification column and containing predominantly acetic acid is recycled to the inlet of the esterification reactor 1.

The recycling of said stream (F₅) is carried out so that the molar ratio of acetic acid to ethanol is 16 at the inlet 0 of the reactor 1. Under these conditions, the ratio by weight of the recycling flow rate (F₅) to the feed flow rate (F₀) is 12.

The stream (F₂) at the column top containing predominantly the ethyl acetate (90% by weight), the water of reaction (8% by weight) and 2.0% by weight of unconverted ethanol is condensed in an exchanger 4 and then sent to a decanter 5 in which two phases are separated.

The aqueous phase (F₆) contains predominantly water and to a minor extent ethanol and ethyl acetate. This aqueous phase (F₆) is sent to a distillation column with a view to separating the water (F₁₆) and a stream comprising predominantly the unconverted ethanol and the ethyl acetate, which stream is recycled (F₁₅) to the esterification reactor.

The organic phase (F₆+F₉) exiting from the decanter 5 contains predominantly ethyl acetate and, at saturation, water, ethanol and traces of acetic acid.

A portion (F₆) of this organic phase is returned to the column top to ensure the reflux with an (F₆)/(F₉) reflux ratio of 3.6.

The other portion (F₉) of the organic phase is subsequently sent to a liquid/liquid extractor 6 operating countercurrentwise at a temperature of 30° C. and at atmospheric pressure.

The ratio of the flow rate of the organic phase to that of the aqueous phase at the start of the liquid/liquid extraction stage is 6. The stream (F₁₀) of water C originates from the stream (F₁₆).

There are obtained, at the outlet of the liquid/liquid extractor 6, a stream (F₁₂) comprising predominantly the water and a stream (F₁₁) comprising predominantly the ester, which stream (F₁₁) is subsequently sent to a distillation column 7 so as to remove the water and the ethanol present in it in order to obtain the quality desired for the final product (F₁₃).

The ethanol content in the ethyl acetate after this final distillation is less than 0.03% by weight.

### Comparative Example 1

### Process without Liquid/Liquid Extraction (CE-1)

The process according to example 1 is reproduced, apart from the liquid/liquid extraction. The portion (F₉) of the organic phase is subsequently directly distilled so as to remove the water and the ethanol present in it in order to obtain the quality desired for the final product.

### Example 2 (E-2)

The above process described in example 1 is reproduced for an acetic acid/alcohol molar ratio of 7 at the inlet 0 of the reactor 1.

### Comparative Example 2 (CE-2)

The above process described in comparative example 1 is reproduced for an acetic acid/alcohol molar ratio of 7 at the inlet 0 of the reactor 1.

### Example 3 (E-3)

The above process described in example 1 is reproduced for an acetic acid/alcohol molar ratio of 25 at the inlet 0 of the reactor 1.

### Comparative Example 3 (CE-3)

The above process described in comparative example 1 is reproduced for an acetic acid/alcohol molar ratio of 25 at the inlet 0 of the reactor 1.

### Example 4 (E-4)

The above process described in example 1 is reproduced for an acetic acid/alcohol molar ratio of 5 at the inlet 0 of the reactor 1.

### Comparative Example 4 (CE-4)

The above process described in comparative example 1 is reproduced for an acetic acid/alcohol molar ratio of 5 at the inlet 0 of the reactor 1.

### Results:

The results are presented in the table below, in which the comparison between the examples is understood to mean with all the other parameters moreover being equal. The overall steam consumption of the whole of the plant was measured for each of the examples.

| Parameters | Unit | E-4 | CE-4 | E-2 | CE-2 | E-1 | CE-1 | E-3 | CE-3 |
|---|---|---|---|---|---|---|---|---|---|
| Acetic | mol/mol | 5 | 5 | 7 | 7 | 16 | 16 | 25 | 25 |
| acid/ethanol | | | | | | | | | |
| ratio | | | | | | | | | |
| (F₅)/(F₀) | kg/kg | 3 | 2.8 | 4.4 | 4.4 | 12 | 12 | 20 | 20 |
| DC ethanol | % | 86.1 | 87.5 | 86.6 | 86.9 | 83.3 | 83.7 | 75.1 | 76.1 |
| Ethanol at the | % by | 3.2 | 3.4 | 2.6 | 2.9 | 1.8 | 2.0 | 1.8 | 1.8 |
| column top (F₂) | weight | | | | | | | | |
| Acetic acid in | ppm | 49 | 50 | 48 | 50 | 50 | 49 | 48 | 50 |
| the stream (F₉) | | | | | | | | | |
| Acetic acid in | ppm | 31 | — | 29 | — | 28 | — | 29 | — |
| the stream (F₁₁) | | | | | | | | | |
| Ethanol after | % by | 2.0 | — | 1.6 | — | 1.1 | — | 1.1 | — |
| extraction | weight | | | | | | | | |
| Ethanol after | % by | 0.03 | 0.6 | 0.03 | 0.03 | 0.03 | 0.03 | 0.03 | 0.03 |
| final distillation | weight | | | | | | | | |
| Overall steam | MWh/t | 1.6 | 2.5 | 1.4 | 2.3 | 1.3 | 1.5 | 1.3 | 1.5 |
| consumption | | | | | | | | | |

It is found that the processes of the examples according to the invention make it possible to reduce the overall steam consumption of the plant (measured over all of the devices of the plant), in comparison with processes without liquid/liquid extraction, all the characteristics moreover being equal.

It is also found that the increase in the acetic acid/ethanol molar ratio at the inlet of the esterification reactor makes it possible to decrease the energy consumption of the column for the final distillation of the ethyl acetate and therefore the overall consumption of the unit.

1. A process for producing a carboxylic acid ester, the process comprising producing the carboxylic acid ester from an organic phase resulting from an esterification reaction between a carboxylic acid and an alcohol, said organic phase comprising at least predominantly the carboxylic acid ester and to a minor extent the alcohol, water and traces of carboxylic acid, wherein the process further comprises a separation stage during which the organic phase is depleted in alcohol and freed from a carboxylic acid by a liquid/liquid extraction operation by bringing said organic phase into contact with an aqueous phase which makes it possible to extract the alcohol and the carboxylic acid present in the organic phase toward the aqueous phase.

2. The process as described by claim 1, wherein in the liquid/liquid extraction an organic phase enriched in carboxylic acid ester, depleted in alcohol and freed from the carboxylic acid is obtained and an aqueous phase comprising predominantly water, alcohol and traces of carboxylic acid is obtained.

3. The process as described by claim 1, wherein the organic phase enriched in carboxylic acid ester, depleted in alcohol and freed from the carboxylic acid has the following composition:
from 85% to 98% by weight,
from 0.5% to 3% by weight,
from 2% to 8% by weight.

4. The process as described by claim 1, wherein the aqueous phase comprising predominantly water, alcohol and traces of carboxylic acid has the following composition:
from 85% to 98% by weight,
from 0.5% to 3% by weight,
from 2% to 8% by weight of carboxylic acid ester, and
traces of carboxylic acid.

5. The process as described by claim 1, wherein the liquid/liquid extraction operation is carried out at a temperature of 15° C. to 50° C., and is carried out at a pressure of from 0.5 bar absolute to 5 bar absolute.

6. The process as described by claim 1, wherein the ratio of the flow rate of the organic phase to the flow rate of the aqueous phase at the start of the liquid/liquid extraction stage is from 2 to 10.

7. The process as described by claim 1, wherein the organic phase comprising at least predominantly the carboxylic acid ester and to a minor extent the alcohol, water and traces of carboxylic acid results from the following stages:
a) an esterification reaction between the carboxylic acid and the alcohol, optionally in the presence of a homogeneous or heterogeneous catalyst,
b) a distillation of the reaction medium resulting from stage a), making it possible to obtain, at the distillation column top, a vapor stream comprising predominantly the carboxylic acid ester and to a minor extent alcohol, water and traces of carboxylic acid,
c) a condensation of the vapor stream resulting from stage b), and
d) a separation of the organic and aqueous phases, optionally by settling.

8. The process as described by claim 1, wherein the organic phase comprises at least predominantly the carboxylic acid ester and to a minor extent the alcohol, water and traces of carboxylic acid results from the following stages:
a′) a reactive distillation between the carboxylic acid and the alcohol, optionally in the presence of a catalyst, making it possible to obtain, at the distillation column top, a vapor stream comprising predominantly the carboxylic acid ester and to a minor extent alcohol and water,
b′) a condensation of the vapor stream resulting from stage a′), and
c′) a separation of the organic and aqueous phases, in optionally by settling.

9. The process as described by claim 7, wherein the alcohol and the carboxylic acid are introduced with a carboxylic acid/alcohol molar ratio of from 0.9 to 25.

10. The process as described by claim 9, wherein the alcohol and the carboxylic acid are introduced with a carboxylic acid/alcohol molar ratio of from 7 to 25.

11. The process as described by claim 1, wherein the aqueous phase which makes it possible to extract the alcohol and the carboxylic acid present in the organic phase toward the aqueous phase is water or originates, in whole or in part, from the aqueous phase resulting from stages d) or c′) which has been subjected to a purification.

12. The process as described by claim 1, wherein the carboxylic acid is an aliphatic carboxylic acid having from 1 to 6 carbon atoms.

13. The process as described by claim 1, wherein the alcohol is selected from the group consisting of ethanol, butanol, n-propanol and cyclohexanol.

14. The process as described by claim 1, wherein the organic phase depleted in alcohol and freed from the carboxylic acid recovered at the outlet of the liquid/liquid extraction stage is subjected to a distillation operation which makes it possible to recover, at the distillation top, the water and the alcohol which could not be removed during the liquid/liquid extraction stage and, at the distillation bottom, the purified carboxylic acid ester.

15. A device for implementing the process as described by claim 1, wherein the device comprises a liquid/liquid extraction device, means for feeding the liquid/liquid extraction device with organic phase comprising predominantly the carboxylic acid ester and to a minor extent the alcohol, water and traces of carboxylic acid, and means for feeding the liquid/liquid extraction device with aqueous phase, and also means for recovery of the aqueous and organic phases at the outlet of the liquid/liquid extraction device.

16. The process as described by claim 3, wherein the organic phase comprises from 90% to 95% by weight of carboxylic acid ester.

17. The process as described by claim 3, wherein the organic phase comprises from 1% to 2% by weight of alcohol.

18. The process as described by claim 3, wherein the organic phase comprises from 3% to 7% by weight of water.

19. The process as described by claim 4, wherein the aqueous phase comprises from 90% to 95% by weight of water.

20. The process as described by claim 4, wherein the aqueous phase comprises from 3% to 5% by weight of alcohol.

21. The process as described by claim 4, wherein the aqueous phase comprises from 4% to 6% by weight of carboxylic acid ester.

22. The process as described by claim 5, wherein the liquid/liquid extraction operation is carried out at a temperature of 20° C. to 40° C.

23. The process as described by claim 6, wherein the ratios of the flow rate of the organic phase to the flow rate of the aqueous phase at the shaft of the liquid/liquid extraction stage is from 4 to 8.

24. The process as described by claim 9, wherein the carboxylic acid/alcohol molar ratio is from 1 to 20.

25. The process as described by claim 10, wherein the carboxylic acid/alcohol molar ratio is from 9 to 20.

26. The process as described by claim 12, wherein the carboxylic acid is acetic acid.

27. The process as described by claim 13, wherein the alcohol is ethanol.
